(19) United States
(12) Patent Application Publication
(10) Pub. No.: US 2009/0064539 A1
(43) Pub. Date: Mar. 12, 2009

(54) HIGH-FUNCTIONAL SHOE HAVING FUNCTION FOR PAIN ALLEVIATION AND PREVENTION OF DEGENERATIVE GONARTHRITIS

(76) Inventors: Hae Soo PARK, Seoul (KR); Chun Kyun NA, Seoul (KR)

Correspondence Address: VIDAS, ARRETT & STEINKRAUS, P.A., SUITE 400, 6640 SHADY OAK ROAD, EDEN PRAIRIE, MN, 55344, US

(21) Appl. No.: 11/960,375

(22) Filed: Dec. 19, 2007

(30) Foreign Application Priority Data
Sep. 6, 2007 (KR) 10-2007-0090272

(51) Int. Cl. A43B 7/14; A43B 13/14; A61F 5/14

(52) U.S. Cl. 36/88; 36/31; 36/144

(57) ABSTRACT

Disclosed therein is a high-functional shoe having functions for a pain alleviation and prevention of degenerative gonarthritis, which can alleviate a pain in the knee joint of a person, who suffers from degenerative gonarthritis or is injured in the knee joint by an abnormal lower limb joint movement, and prevent degenerative gonarthritis by uniformly dispersing a load biasedly applied to the knee joint. The high-functional shoe includes a shoe sole having a longitudinally sectional structure of a parallelogrammic form, and is applicable to persons, who have symptoms of degenerative gonarthritis.

### BACKGROUND OF THE INVENTION

1. Field of the Invention

The present invention relates to a high-functional shoe having functions for a pain alleviation and prevention of degenerative gonarthritis, which can alleviate a pain in the knee joint of a person, who suffers from degenerative gonarthritis or is injured in the knee joint by an abnormal lower limb joint movement, and prevent degenerative gonarthritis by uniformly dispersing a load biasedly applied to the knee joint.

2. Background Art

In general, the knee joint is a part to keep the balance in such a way that a human being can bear weight of the body, which must be born with four feet, with two feet to thereby stand up without falling down. So, the knee joint keeps the balance of the body while moving little by little even in the moment that we cannot be aware thereof. However, in case of most of people, since the center of weight does not pass through the center of the knee joint, a load heavier than a compressive force by the body weight continuously acts on a portion of the knee joint. So, the knee joint is a part where a degenerative change is generated the most as the human being grows older. Besides the above, degenerative arthritis occurs due to excessive exercises or overweight. In addition, meniscus injuries that semilunar cartilage of the knee is cracked by an trauma, a ligament injury generated by a strong shock or an excessive rotation of the knee joint, chondromalacia patella frequently (generally) generated to young women, and bursitis that mucous cyst of the knee joint is inflamed are diseases frequently generated on the knee joint.

Degenerative gonarthritis shows symptoms of that a patient has a pain in the knee when he or she tries to stand up, the knee is trembled when the patient stands up, a sound is produced when the patient bends or spreads out the knee, or the legs become open as wide as at least one fist goes between the knees in an erect posture. Moreover, degenerative gonarthritis is a disease, which is open to doubt when the pain is specially severe when the patient goes downstairs or when the pain generated after walking is continued for more than 2-3 days.

Joints are moved under a load, and particularly, joints of legs, such as the knee joints, receive load, which is several times of the body weight, by ordinary motions, such as standing, walking, ascending stairs, and so on. The knee joint receives the strongest load out of various joints in the body.

Degenerative arthritis is generally generated from fifties, but gonarthritis is generated from forties, which is a relatively early time. A joint, which has a wound or a disease, may be accompanied with an edema, a pain or a symptom that the joint gets stiff.

An object to cure arthritis is to alleviate the pain and increase pliability of the joint. However, in case of most of arthritis diseases, it is impossible to be recovered completely. In case of a slight symptom, the pain and the stiff symptom can be alleviated through a continuous physical therapy, a medicinal therapy, reduction of weight, and so on. However, in case of a severe symptom of arthritis, the patient can reduce the pain through a surgical operation or recover the joint function through reconstruction.

For instance, total knee replacement is a method to remove a knee joint, which has a severe arthritis, and transplant a new joint. The patient, who has gonarthritis, must continuously undergo such a medical treatment to be released from the pain. However, a great deal of patients cannot undergo the medical treatment properly and suffer from the pain due to inconvenience and high cost for the medical treatment.

FIG. 1 is an anatomically structural diagram of the lower limbs of a human body, which is viewed from the front, and FIG. 2 is a view showing a posture that a human being stands on the ground with one foot during walking. As shown in FIGS. 1 and 2, since a weight central line 10 of the human body goes through the center between two legs 2 when the human being 1 stands on the ground 3 with two legs 2, a more severe compressive load is applied to the inside than the outside of the knee joint. However, when the human being 1 stands on the ground 3 with just one leg 2, by pronation, the load acting to the inside of the knee 2 is increased more than when the human being 1 stands on the ground 3 with the two legs 2. Pronation means a phenomenon that a person's foot and shin rotate inwardly, but supination means a phenomenon that a person's foot and shin rotate outwardly.

FIG. 3 is a view showing the pronation generated when the human being stands on the ground with one foot, FIGS. 4 and 5 are mechanical diagrams of the supination generated during walking, and FIGS. 6 and 7 are mechanical diagrams of the pronation generated during walking. As shown in FIG. 3, when the pronation is generated while the human being stands on the ground 3 with just one foot 2, the shin and the thigh are rotated together in an arrow direction 4, so that a load is applied to the inner knee joint, such motions are repeatedly generated during walking.

As shown in FIGS. 4 and 5, in which a skeletal structure formed between the shin of the leg 2 and the foot is illustrated mechanically, when the supination is generated during walking, the shin 20 is rotated in an arrow direction 22 but the foot 30 is rotated in another arrow direction 32. That is, the shin 20 and the foot 30 are all rotated outwardly.

As shown in FIGS. 6 and 7, when the pronation is generated during walking, the shin 20 is rotated in an arrow direction 22′ but the foot 30 is rotated in another arrow direction 32′. That is, the shin 20 and the foot 30 are all rotated inwardly.

If the mechanical phenomenon is repeated, the human being must take an exercise for strengthening the lower limb muscle power from the forties. Otherwise, since the legs are transformed into an “O”-shaped legs and the range of motion in dorsiflexion of an ankle is also reduced, it is difficult to walk energetically and a pace and a stride are reduced.

Particularly, in case of women, if they are over sixty-five, most of them feel inconvenient in walking and walk bowlegged since their legs are bent in the “O” shape. In case of the women, due to childbirth, a life pattern which overdrives the knee joint, and a physiological change by menstruation, they are lower in an outbreak age of gonarthritis and higher in frequency of outbreak of gonarthritis than man. On the other hand, man is less in the case of “O”-shaped legs than women, and obtains the “O”-shaped legs in an age group older than women.

As you can see from the clinical observation that degenerative gonarthritis is easily and mainly broken out in the inner front portion and the knee is transformed into the “O”-shaped leg relatively from the forties, it could be analyzed that degenerative gonarthritis is broken out when a load is biasedly concentrated on the knee joint for a long time. In addition, it can be recognized that degenerative gonarthritis can be prevented and the pain of degenerative gonarthritis can be alleviated just through a dispersion of the load biasedly concentrated on the knee joint.

Deterioration in degenerative functions and injury of the knee joint are generated by an injury due to a sudden traumatic injury, an anatomic lower limb structure, or high-heeled shoes.

Based on a close examination of the above causes, there is a method to adjust a skeletal structure and equalize a load distribution by changing shoe sole form. Moreover, there is another method to transfer the center of gravity, which was transferred to the front of the body when the patient wears high-heeled shoes, to the neutral position or the rear position, which is the same as barefoot walking. Through the above methods, the pain of the knee joint can be alleviated without undergoing the surgical operation, the physical treatment or the medicinal therapy. Furthermore, it is possible to develop a functional shoe for alleviating the pain of gonarthritis and preventing gonarthritis, which can help the patient to take his or her daily life or sports activities to increase pliability of joints and reinforce muscular strength.

The method for alleviating the pain of degenerative gonarthritis and preventing degenerative gonarthritis using the shoe is nearly similar with the method used for correcting the “O”-shaped legs and for adjusting the excessive supination in aspects of the outbreak causes and symptoms. In the typical orthopedic shoemaking field, a method of adding a wedge on the outer surface of a shoe liner or on a shoe sole is employed. That is, it is a method to incline a wearer's legs inwardly and cause a rotational force for transferring the knees inwardly according to the slope of the shoe liner or the shoe sole when the wearer wears the shoes and steps on the ground.

However, such a method generates a shear stress since the wearer's foot is slid inwardly due to the slope of the shoe sole. Moreover, when the wearer excessively devotes his or her strength to the leg to prevent a slide of the foot, a strong load is applied to the inner portion of the foot and the knee to thereby make degenerative gonarthritis worse.

Because of such side effects, the orthopedic shoemaking shall observe the doctor's prescription for each customer. When inclination angles are not accurately matched with each other since the orthopedic shoe is made without observing the doctor's prescription for each customer, severe side effects may be caused due to the shear stress.

So, a new method, which can disperse the biasedly concentrated load without generating the shear stress and without causing side effects on the musculoskeletal system even though the shoe form is not exactly matched with the wearer's leg form, is needed. In addition, it is also needed that the patient, who has degenerative gonarthritis, can conveniently buy the shoe in a general shop and wear them conveniently without seeing a doctor and ordering custom-made shoes according to the doctor's prescription.

### SUMMARY OF THE INVENTION

Accordingly, the present invention has been made to solve the above-mentioned problems occurring in the prior arts, and it is an object of the present invention to provide a high-functional shoe having functions for a pain alleviation and prevention of degenerative gonarthritis, which includes a shoe sole formed in a parallelogram to prevent a wearer's foot from sliding inwardly or outwardly, thereby preventing a generation of a shear stress.

It is another object of the present invention to provide a high-functional shoe having functions for a pain alleviation and prevention of degenerative gonarthritis, which can generate a rotational force to reduce pressure on a painful knee portion, thereby alleviating a pain of degenerative gonarthritis.

It is a further object of the present invention to provide a high-functional shoe having functions for a pain alleviation and prevention of degenerative gonarthritis, which can prevent degenerative gonarthritis when a normal person wears the shoe.

It is a still further object of the present invention to provide a high-functional shoe having functions for a pain alleviation and prevention of degenerative gonarthritis, which can correct a leg deformation by varying a slope of the parallelogrammic shoe sole according to individuals, who have different lower limb skeletal alignments.

It is another object of the present invention to provide a high-functional shoe having functions for a pain alleviation and prevention of degenerative gonarthritis, which can keep a balance of the body and induce a safe walking.

To accomplish the above objects, according to the present invention, there is provided a high-functional shoe having functions for a pain alleviation and prevention of degenerative gonarthritis, which includes a shoe sole and an upper, wherein the shoe sole has a longitudinally sectional structure of a parallelogrammic form so that the high-functional shoe is applicable to a person, who has symptoms of degenerative gonarthritis, to thereby alleviate the pain of degenerative gonarthritis and prevent degenerative gonarthritis.

In addition, the shoe sole has an inclination angle inclined to the outside in regard to a vertical line of the inside of the shoe, and is applicable to a person, who has a pain mainly in the inside of the knee, who has the legs under transformation into “O”-shaped legs, or who has the “O”-shaped legs.

Moreover, the shoe sole has an inclination angle inclined to the inside in regard to a vertical line of the outside of the shoe, and is applicable to a person, who has a pain mainly in the outside of the knee, who has the legs under transformation into “X”-shaped legs, or who has the “X”-shaped legs.

Furthermore, the inclination angle of the shoe sole is equal to an angle formed between a femur inclination axis and a mechanical axis.

Additionally, the inclination angle is any one selected from 0°, 3°, 5°, 7°, 10° and 15°.

Moreover, the high functional shoe further includes a wedge member mounted on an inner sole or the shoe sole in such a way that the foot is inclined inwardly, and being applicable to a person, who has a pain mainly in the inside of the knee, who has the legs under transformation into “O”-shaped legs, or who has the “O”-shaped legs.

In addition, the high functional shoe further includes a wedge member mounted on an inner sole or the shoe sole in such a way that the foot is inclined outwardly, and being applicable to a person, who has a pain mainly in the outside of the knee, who has the legs under transformation into “X”-shaped legs, or who has the “X”-shaped legs.

Furthermore, in another aspect of the present invention, the present invention provides a high-functional shoe having functions for a pain alleviation and prevention of degenerative gonarthritis, the high-functional shoe comprising: an upper; an inner sole constructed such that the foot is inclined to one side; and a shoe sole adapted to be inclined such that its longitudinal cross-sectional structure becomes a parallelogram when the shoe sole is coupled with the inner sole, thereby making the shoe applicable to a person, who has symptoms of degenerative gonarthritis.

Additionally, the inner sole is inclined in such a way that the foot is inclined to the outside for a person, who has a pain mainly in the inside of the knee, who has the legs under transformation into “O”-shaped legs, or who has the “O”-shaped legs.

Moreover, the inner sole is inclined in such a way that the foot is inclined to the inside for a person, who has a pain mainly in the outside of the knee, who has the legs under transformation into “X”-shaped legs, or who has the “X”-shaped legs.

As described above, the high-functional shoe according to the present invention can alleviate the pain of degenerative gonarthritis and prevent gonarthritis by uniformly dispersing the load biasedly applied to the knees when the wearer wears the shoe.

In addition, in case of “O”-shaped or “X”-shaped legs, each of which has a lower limb skeletal alignment deviating from normality, high-functional shoes with different forms according to leg forms of the wearers can be manufactured using the parallelogrammic shoe sole and a technology, which has been typically used in the orthopedic shoemaking field, together. Furthermore, the high-functional shoe according to the present invention can double the effect of correcting the deformation in leg alignment since the wearer can wear the shoe fit to his or her leg form.

Additionally, the high-functional shoe according to the present invention can allow the wearer to establish a correct walking posture and a correct body structure as well as correct the leg deformation.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is an anatomically structural view of the lower limb viewed from the front;

FIG. 2 is a view showing a state where a wearer stands on the ground with one foot;

FIG. 3 is a view showing pronation generated when the wearer stands on the ground with one foot;

FIGS. 4 and 5 are mechanical diagrams showing supination generated during walking;

FIGS. 6 and 7 are mechanical diagrams showing pronation generated during walking;

FIG. 8 is a front view of the right lower limb alignment of an “O”-shaped legs;

FIG. 9 is a front view of the right lower limb alignment of a “X”-shaped legs;

FIG. 10 is a rear view of a shoe for a person, who has symptoms of degenerative arthritis in the knee and has a pain mainly in the inside of the knee or has the legs under transformation into the “O”-shaped legs, according to a first preferred embodiment of the present invention;

FIG. 11 is a rear view of the shoe of FIG. 10, to which a wedge is added;

FIG. 12 is a rear view of a shoe for a person, who has symptoms of degenerative arthritis in the knee and has a pain mainly in the outside of the knee or has the legs under transformation into the “X”-shaped legs, according to the first preferred embodiment of the present invention;

FIG. 13 is a rear view of the shoe of FIG. 12, to which a wedge is added;

FIG. 14 is a rear view of a shoe for a person, who has symptoms of degenerative arthritis in the knee and has a pain mainly in the inside of the knee or has the legs under transformation into the “O”-shaped legs, according to a second preferred embodiment of the present invention;

FIG. 15 is a rear view of a shoe for a person, who has symptoms of degenerative arthritis in the knee and has a pain mainly in the outside of the knee or has the legs under transformation into the “X”-shaped legs, according to the second preferred embodiment of the present invention.

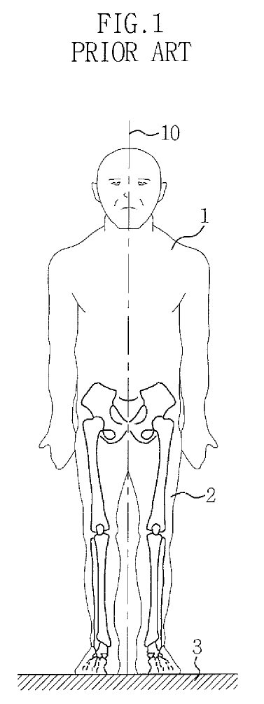

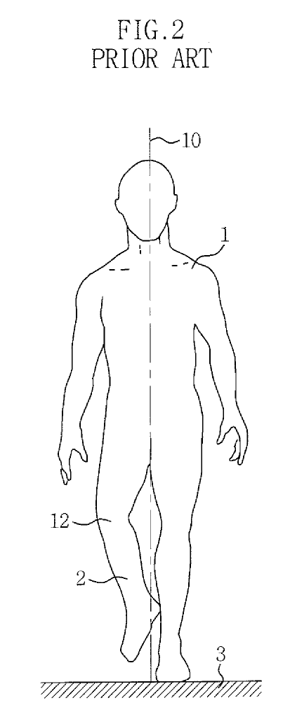

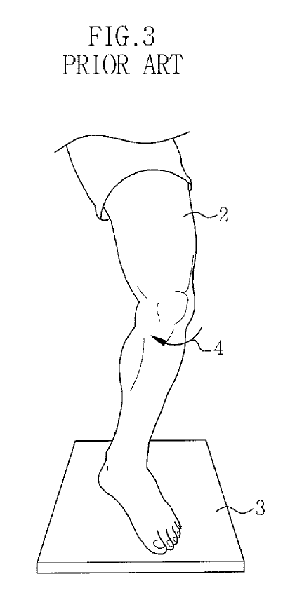

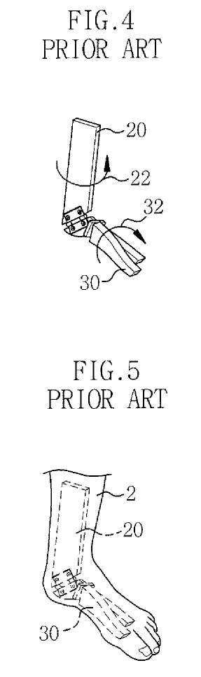

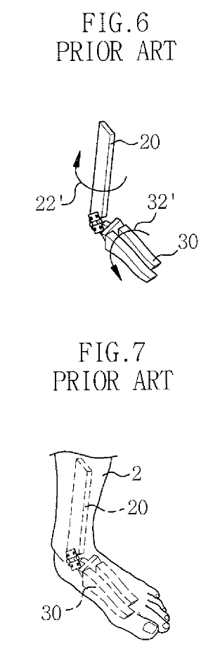

### DETAILED DESCRIPTION OF THE PREFERRED EMBODIMENT

Reference will be now made in detail to the preferred embodiment of the present invention with reference to the attached drawings. In the drawings, the same components have the same reference numerals even though they are illustrated on different drawings. Furthermore, if it is judged that detailed descriptions of relevant well-known functions or structures may make obscure the essential points of the present invention, the detailed descriptions will be omitted.

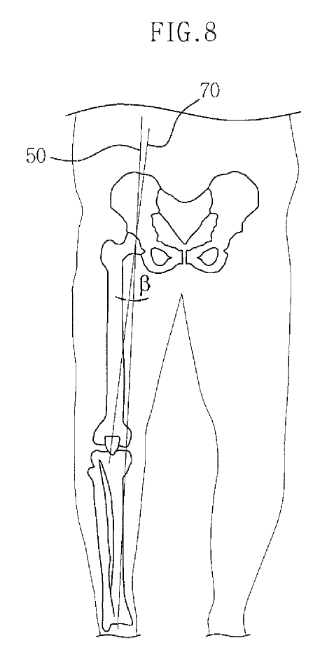

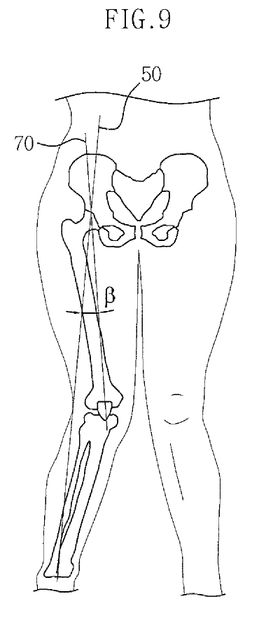

FIG. 8 is a front view of the lower limb alignment of the right knee of “O”-shaped legs, and FIG. 9 is a front view of the lower limb alignment of the right knee of “X”-shaped legs. As shown in FIGS. 8 and 9, a mechanical axis 50 means a line extending from the center of the head of femur to the center of the head of talus, and a femur inclination axis 70 means a line extending from the center of the head of femur to the center of a knee joint.

Genu varum generally called the “O”-shaped legs means a case that the femur inclination axis 70 is inclined to the outside of the mechanical axis 50 Genu valgum generally called the “X”-shaped legs means a case that the femur inclination axis 70 is inclined to the inside of the mechanical axis 50. If a value of an inclination angle (β) formed between the femur inclination axis 70 and the mechanical axis 50 is large, it means that the extent of the “O”-shaped legs or the “X”-shaped legs is severe.

In the description of the present invention, the persons, who have the “O”-shaped legs include all of persons, who have the “O”-shaped lower limb alignment congenitally, and persons, whose lower limb alignment is transformed into the “O” shape acquiredly. In addition, the persons, who have the “X”-shaped legs include all of persons, who have the “X”-shaped lower limb alignment congenitally, and persons, whose lower limb alignment is transformed into the “X” shape acquiredly.

The shoe described in this description designates the right shoe, to which the present invention is applied, but the present invention can be applied to the left shoe in the same way as the right shoe.

FIG. 10 is a rear view of a shoe for a person, who has symptoms of degenerative arthritis in the knee and has a pain mainly in the inside of the knee or has the legs under transformation into the “O”-shaped legs, according to a first preferred embodiment of the present invention.

As shown in FIG. 10, a shoe sole 140 is formed in a parallelogram in a longitudinally sectional structure.

A shoe 100 or 200 includes the upper 120 or 220 surrounding foot, and a shoe sole 140 or 240 for supporting the foot. The shoe sole 140 or 240 is manufactured in such a way as to be inclined at an angle of α to thereby have a longitudinally sectional structure of a parallelogram, so that it can be worn differently according to a wearer's body portion, which has degenerative gonarthritis, and a lower limb skeletal alignment form.

### First Embodiment

As shown in FIG. 10, the shoe sole 140 has an inclination angle of α in an outward direction 180 to a vertical line of the inside 170 of the shoe. In this instance, the shoe sole 140 is coupled with the upper 120 in such a way that the upper and lower faces of shoe sole are horizontal with each other.

Since the shoe sole 140 has the inclination angle α in the outward direction 180, the center of the sole of the foot deviates from the center of the shoe sole. That is, since the center of the sole of the foot is located inwardly from the center of the shoe sole 140, a joint clearance of the inside of the knee is widened due to a generation of the moment, and thereby, a load is reduced. Moreover, an interval between the right and left knees becomes narrow, and the knees are corrected inwardly.

In this instance, since the shoe sole 140 is in contact with the ground 160 horizontally, also the sole of the foot keeps horizontality with the ground 160 to thereby prevent a generation of a shear stress. Furthermore, since the load concentrated on the inside of the knee is reduced and is dispersed to the whole knee, if the person, who has the symptoms of degenerative gonarthritis in the inside of the knees or whose legs are transformed into the “O”-shaped legs congenitally or acquiredly, wears the shoe, he or she can walk in stable since the pain of degenerative gonarthritis is alleviated.

In addition, even in case of a person, who does not have the symptoms of degenerative gonarthritis or has a slight symptom of degenerative gonarthritis, if the person continuously wears the shoe 100 having the shoe sole 140 of the parallelogrammic form, it can prevent degenerative gonarthritis since the pressure concentrated on the inside of the knee during walking is reduced and is dispersed to the whole knee. In this instance, the person, who has a normal lower limb skeletal alignment, wears the shoe sole 140 having an inclination angle smaller than the inclination angle (α) applied to the person, who has the “O”-shaped legs.

Furthermore, even in case of a person, who does not have the symptoms of degenerative gonarthritis but has the “O”-shaped legs, if the person continuously wears the shoe 100 having the shoe sole 140 of the parallelogrammic form, it can correct the “O”-shaped legs into “|”-shaped legs, which are normal legs, and prevent degenerative gonarthritis.

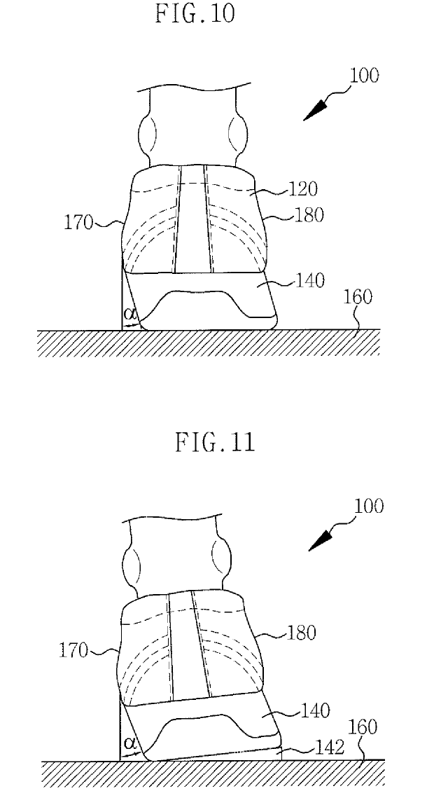

FIG. 11 is a rear view of the shoe of FIG. 10, to which a wedge is added. As shown in FIG. 11, the shoe may further include a wedge member 142 located on an inner sole of the shoe 100 or on the bottom surface of the shoe sole 140 in such a way as to make the foot more inclined in the inward direction 170.

In case that the symptoms of gonarthritis are severe or the legs are greatly transformed into the “O”-shaped legs, the wedge member 142 is coupled to the parallelogrammic shoe sole 140 or a shoe sole or an inner sole, which is typically used in the orthopedic shoemaking field, to reduce more the load acting to the inside of the knee and enhance the effect of correcting the knee.

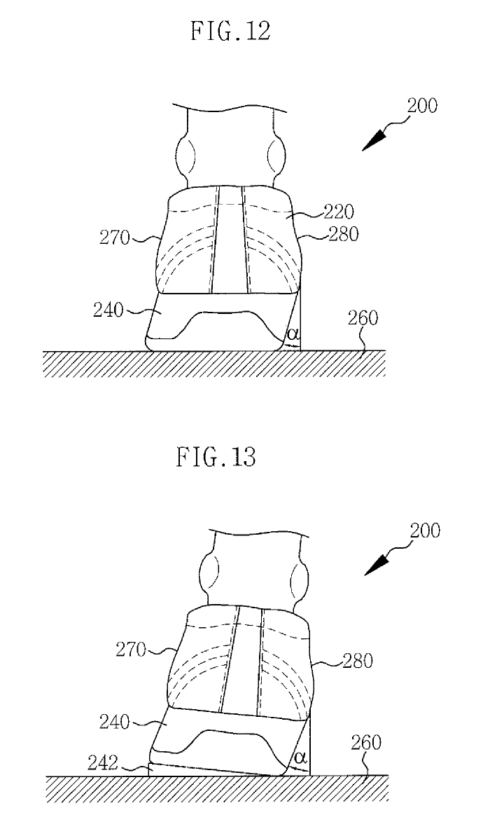

FIG. 12 is a rear view of a shoe for a person, who has symptoms of degenerative arthritis in the knee and has a pain mainly in the outside of the knee or has the legs under transformation into the “X”-shaped legs, according to the first preferred embodiment of the present invention. As shown in FIG. 12, the shoe sole 240 has an inclination angle of a in an inward direction 270 to a vertical line of the outside 280 of the shoe. In this instance, the shoe sole 240 is coupled with the upper 220 in such a way that the upper and lower faces of shoe sole are horizontal with each other.

Since the shoe sole 240 has the inclination angle α in the inward direction 270, the center of the sole of the foot deviates from the center of the shoe sole. That is, since the center of the sole of the foot is located outwardly from the center of the shoe sole 240, a joint clearance of the outside of the knee is widened due to a generation of the moment, and thereby, a load is reduced. Moreover, an interval between the right and left knees is widened, and the knees are corrected outwardly.

In this instance, since the shoe sole 240 is in contact with the ground 260 horizontally, also the sole of the foot keeps horizontality with the ground 160 to thereby prevent a generation of a shear stress. Furthermore, since the load concentrated on the outside of the knee is reduced and is dispersed to the whole knee, if the person, who has the symptoms of degenerative gonarthritis in the outside of the knee or whose legs are transformed into the “X”-shaped legs congenitally or acquiredly, wears the shoe, he or she can walk in stable since the pain of degenerative gonarthritis is alleviated.

Furthermore, even in case of a person, who does not have the symptoms of degenerative gonarthritis but has the “X”-shaped legs, if the person continuously wears the shoe 200 having the shoe sole 240 of the parallelogrammic form, it can correct the “X”-shaped legs into “|”-shaped legs, which are normal legs, and prevent degenerative gonarthritis.

FIG. 13 is a rear view of the shoe of FIG. 12, to which a wedge is added. As shown in FIG. 13, the shoe may further include a wedge member 242 located on an inner sole of the shoe 200 or on the bottom surface of the shoe sole 240 in such a way as to make the foot more inclined in the outward direction 280.

In case that the symptoms of gonarthritis are severe or the legs are greatly transformed into the “X”-shaped legs, the wedge member 242 is coupled to the parallelogrammic shoe sole 240 or a shoe sole or an inner sole, which is typically used in the orthopedic shoemaking field, to reduce more the load acting to the outside of the knee and enhance the effect of correcting the knee.

In addition, in case of a shoe, which the person having the “O”-shaped legs or the “X”-shaped legs wears, an angle (β) formed between the femur inclination axis 70 and the mechanical axis 50 is equal to the inclination angle (α) that the shoe sole 140 or 240 is inclined inwardly or outwardly.

Moreover, since people are different from one another in a leg form and a curved level of the knee, people are also different from one another in the angle (β) formed between the femur inclination axis 70 and the mechanical axis 50. So, the angle formed between the femur inclination axis 70 and the mechanical axis 50 is grasped using an X-ray examination, and then, the shoe sole 140 or 240 inclined in the same inclination angle as the angle between the femur inclination axis 70 and the mechanical axis 50 can be manufactured.

Furthermore, the shoe sole 140 may be formed integrally and then the inclination angle (α) can be adjusted by inserting or removing a prosthesis to or from the inside of the shoe.

Additionally, it is preferable that the inclination angle (α) of the shoe sole 140 does not exceed 15°. If shoe soles 140, which have been previously produced at predetermined angles, (the value of α has a predetermined difference in angle, for instance, 0°, 3°, 5°, 7°, 10°, 15°, and so on) according to relative seriousness of the symptoms of arthritis, are on market, consumers can select and wear the shoe according to the symptoms of arthritis and the human body's clinical receptivity.

### Second Embodiment

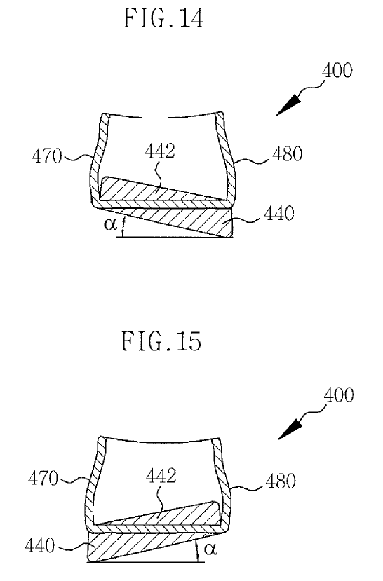

FIG. 14 is a sectional view of a shoe for a person, who has symptoms of degenerative arthritis in the knee and has a pain mainly in the inside of the knee or has the legs under transformation into the “O”-shaped legs, according to a second preferred embodiment of the present invention. FIG. 15 is a sectional view of a shoe for a person, who has symptoms of degenerative arthritis in the knee and has a pain mainly in the outside of the knee or has the legs under transformation into the “X”-shaped legs, according to the second preferred embodiment of the present invention. As shown in FIGS. 14 and 15, a shoe 400 includes: an inner sole 442 constructed such that the foot is inclined to one side; and a shoe sole 440 inclined in such a way that its longitudinally sectional structure becomes a parallelogram when the shoe sole 440 is coupled with the inner sole 442.

In the present invention, the inner sole 442 and the shoe sole 440 are constructed in such a way as to have the longitudinally sectional structure of a triangle type, but the longitudinally sectional structure may be formed in a trapezoid or a parallelogram. In this instance, the longitudinal section formed when the inner sole 442 and the shoe sole 440 are coupled with each other must be in a parallelogrammic form.

Like the first preferred embodiment, in case of the person, who has a pain mainly in the inside of the knee or has the legs under transformation into the “O”-shaped legs, as shown in FIG. 14, the inner sole 442 is constructed such that the foot is inclined to the outside 480.

In addition, in case of the person, who has a pain mainly in the outside of the knee or has the legs under transformation into the “X”-shaped legs, as shown in FIG. 15, the inner sole 442 is constructed such that the foot is inclined to the inside 370. The inner sole 442 is coupled with the shoe sole 440 manufactured in such a way as to form a parallelogram when the inner sole 442 and the shoe sole 440 are coupled with each other.

The inner sole 442 inclined to the inside 470 or the outside 480 puts within the shoe, and the shoe sole 440, which has the longitudinally sectional structure of the parallelogrammic form when the shoe sole 440 is coupled with the inner sole 442, is located on the bottom of the shoe 400. So, the present invention can alleviate the pain of degenerative gonarthritis and prevent degenerative gonarthritis since it does not generate the shear stress like the parallelogrammic shoe sole having the inclination angle (α).

While the present invention has been described with reference to the particular illustrative embodiment, it is not to be restricted by the embodiment but only by the appended claims It is to be appreciated that those skilled in the art can change or modify the embodiment without departing from the scope and spirit of the present invention.

1. A high-functional shoe having functions for a pain alleviation and prevention of degenerative gonarthritis, which includes a shoe sole and an upper, wherein the shoe sole has a longitudinally sectional structure of a parallelogrammic form so that the high-functional shoe is applicable to a person, who has symptoms of degenerative gonarthritis, to thereby alleviate the pain of degenerative gonarthritis and prevent degenerative gonarthritis.

2. The high-functional shoe according to claim 1, wherein the shoe sole has an inclination angle inclined to the outside in regard to a vertical line of the inside of the shoe, and is applicable to a person, who has a pain mainly in the inside of the knee, who has the legs under transformation into “O”-shaped legs, or who has the “O”-shaped legs.

3. The high-functional shoe according to claim 1, wherein the shoe sole has an inclination angle inclined to the inside in regard to a vertical line of the outside of the shoe, and is applicable to a person, who has a pain mainly in the outside of the knee, who has the legs under transformation into “X”-shaped legs, or who has the “X”-shaped legs.

4. The high-functional shoe according to claim 2 or 3, wherein the inclination angle of the shoe sole is equal to an angle formed between a femur inclination axis and a mechanical axis.

5. The high-functional shoe according to claim 4, wherein the inclination angle is any one selected from 0°, 3°, 5°, 7°, 10°and 15°.

6. The high-functional shoe according to claim 2, further comprising a wedge member mounted on an inner sole or the shoe sole in such a way that the foot is inclined inwardly, and being applicable to a person, who has a pain mainly in the inside of the knee, who has the legs under transformation into “O”-shaped legs, or who has the “O”-shaped legs.

7. The high-functional shoe according to claim 3, further comprising a wedge member mounted on an inner sole or the shoe sole in such a way that the foot is inclined outwardly, and being applicable to a person, who has a pain mainly in the outside of the knee, who has the legs under transformation into “X”-shaped legs, or who has the “X”-shaped legs.

8. A high-functional shoe having functions for a pain alleviation and prevention of degenerative gonarthritis, the high-functional shoe comprising:
an upper;
an inner sole constructed such that the foot is inclined to one side; and
a shoe sole adapted to be inclined such that its longitudinal cross-sectional structure becomes a parallelogram when the shoe sole is coupled with the inner sole, thereby making the shoe applicable to a person, who has symptoms of degenerative gonarthritis.

9. The high-function shoe according to claim 8, wherein the inner sole is inclined in such a way that the foot is inclined to the outside for a person, who has a pain mainly in the inside of the knee, who has the legs under transformation into “O”-shaped legs, or who has the “O”-shaped legs.

10. The high-function shoe according to claim 8, wherein the inner sole is inclined in such a way that the foot is inclined to the inside for a person, who has a pain mainly in the outside of the knee, who has the legs under transformation into “X”-shaped legs, or who has the “X”-shaped legs.
